(19) United States
(12) Patent Application Publication
(10) Pub. No.: US 2019/0076379 A1
(43) Pub. Date: Mar. 14, 2019

(54) METHODS OF TREATING BREAST CANCER

(71) Applicant: Mitchell Woods Pharmaceuticals, Inc., Shelton, CT (US)

(72) Inventor: Irving W. Wainer, Washington, DC (US)

(21) Appl. No.: 16/190,821

(22) Filed: Nov. 14, 2018

Related U.S. Application Data
(63) Continuation of application No. 15/300,659, filed on Sep. 29, 2016, now abandoned.
(60) Provisional application No. 61/974,842, filed on Apr. 3, 2014.

(51) Int. Cl. A61K 31/137; A61K 31/704; A61K 45/06

(52) U.S. Cl. CPC A61K 31/137; A61K 45/06; A61K 31/704

(57) ABSTRACT

Methods of regulating disorders and diseases in cells expressing β2-adrenergic receptor (AR), cannabinoid (CB) receptor and epidermal growth factor receptor (EGFR), include treating a disorder or disease, such as a breast cancer include administration of a fenoterol analogue.

### CROSS-REFERENCE TO RELATED APPLICATIONS

The present application is a continuation application of U.S. patent application Ser. No. 15/300,659 filed Sep. 29, 2016, which is a U.S. National Stage Application under 35 U.S.C. § 371 of International Application Number PCT/US2015/023804, filed on Apr. 1, 2015, which claims the benefit of, and priority to, U.S. Provisional Patent Application No. 61/974,842, filed Apr. 3, 2014, the entire content of each of which is incorporated by reference herein.

### TECHNICAL FIELD

The present disclosure relates to methods of regulating disorders and diseases in cells expressing β2-adrenergic receptor (AR), cannabinoid (CB) receptor and epidermal growth factor receptor (EGFR), including treating a disorder or disease, such as a breast cancer by administration of fenoterol analogues.

### BACKGROUND

Cancer is the second leading cause of human death next to coronary disease in the United States. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise. Cancer is soon predicted to become the leading cause of death.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

This disclosure concerns the discovery that fenoterol analogues can be used to treat cancer associated with β2-adrenergic receptor (AR) expression, cannabinoid (CB) receptor expression, and epidermal growth factor receptor (EGFR) expression. The exemplary methods described herein can be used to treat a tumor expressing β2-adrenergic receptor (AR), cannabinoid (CB) receptor and epidermal growth factor receptor (EGFR).

In embodiments, the method includes administering a therapeutically effective amount of a fenoterol analogue to treat a tumor expressing β2-adrenergic receptor (AR), cannabinoid (CB) receptor and epidermal growth factor receptor (EGFR).

Fenoterol analogues useful in connection with the present disclosure include: (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol and their respective stereoisomers.

In embodiments, the fenoterol analogue is (R,R′)-4′-methoxy-1-naphthyl-fenoterol (MNF), a compound having the formula:

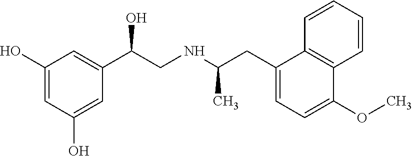 [SMILES: COc1ccc(C[C@@H](C)NC[C@H](O)c2cc(O)cc(O)c2)c2ccccc12]

In embodiments, the presently described methods include administering a therapeutically effective amount of a pharmaceutical composition containing a fenoterol analogue described herein and a pharmaceutically acceptable carrier to treat a tumor expressing β2-adrenergic receptor (AR), cannabinoid (CB) receptor and epidermal growth factor receptor (EGFR), such as a breast cancer expressing β2-adrenergic receptor (AR), a cannabinoid (CB) receptor and epidermal growth factor receptor (EGFR). For example, the fenoterol analogues described herein are effective at treating breast cancer expressing β2-adrenergic receptor (AR), a cannabinoid (CB) receptor and epidermal growth factor receptor (EGFR). In embodiments, the method further includes selecting a subject having or at risk of developing a tumor associated with β2-adrenergic receptor (AR) expression, a cannabinoid (CB) receptor expression, and epidermal growth factor receptor (EGFR) expression. For example, a subject is selected for treatment by determining that the tumor expresses β2-adrenergic receptor (AR), a cannabinoid (CB) receptor and epidermal growth factor receptor (EGFR). In embodiments, the method includes administering one or more therapeutic agents in addition to a fenoterol analogue. The methods can include administration of the one or more therapeutic agents separately, sequentially or concurrently, for example in a combined composition with a fenoterol analogue.

According to exemplary methods described herein, administration of fenoterol analogues inhibits proliferation of tumor cells expressing β2-adrenergic receptor (AR), cannabinoid (CB) receptor and epidermal growth factor receptor (EGFR) such as human breast cancer cells and, therefore, is active in the prevention of tumor metastasis.

Further scope of applicability of the present disclosure will become apparent from the detailed description given hereinafter. However, it should be understood that the detailed description and specific examples, while indicating specific embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

FIGS. 1 and 2 show the effect of MNF on basal phosphorylation levels of ERK in MDA-MB-231 Cells;

FIGS. 3A and 3B show the effect of MNF on the cell cycle of MCF-7 cells and MDA-MB-231 cells;

FIGS. 4A and 4B show the effect of MNF on expression of the cell cycle regulatory proteins Cyclin D1 and Cyclin B1 in MDA-MB-231 cells and MCF-7 cells;

FIG. 5 shows that MNF reduces the expression of Multidrug resistance protein (Mdr) in MCF-7 cells;

FIG. 6 shows that the Combination of Doxorubicin and MNF enhances the anti-proliferative property in MCF-7 cells;

FIG. 7 shows that MNF inhibited the levels of [3H]thymidine incorporation in MDA-MB-231 cells and MCF-7 cells;

FIG. 7 shows cellular proliferation and adherence in UACC-647, M93-047 and UACC-903 cells stimulated with MNF; and

FIG. 8 shows that the inhibitory effect of MNF is blocked by GPR55 agonist, AM251 but not by β2-AR antagonist, ICI 118,551.

The figures depict specific embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

### Introduction

Fenoterol, 5-[1-hydroxy-2 [[2-(4-hydroxyphenyl)-1-methylethyl]-amino] ethyl] 1,2-benzenediol, is a β2-AR agonist that has traditionally been used for the treatment of pulmonary disorders such as asthma. This drug has two chiral (asymmetric) carbons that can each be independently arranged in an R or S configuration, so that the drug exists in distinct (R,R), (R,S), (S,R) and (S,S) forms known as stereoisomers. Fenoterol is commercially available as a racemic mixture of the (R,R)- and (S,S)-compounds.

Fenoterol acts as an agonist that binds to and activates the β2-AR. This activity has led to its clinical use in the treatment of asthma because this agonist's activity dilates constricted airways.

In accordance with the present disclosure, fenoterol analogues are used to treat tumors that express β2-adrenergic receptor (AR), a cannabinoid (CB) receptor and epidermal growth factor receptor (EGFR). In embodiments, the optically active fenoterol analogues are substantially purified from a racemic mixture. For example, an optically active fenoterol analogue is purified to represent greater than 90%, often greater than 95% of the composition. These analogues can be used to treat a tumor that expresses β2-adrenergic receptor (AR), a cannabinoid (CB) receptor and epidermal growth factor receptor (EGFR). It is specifically contemplated that the fenoterol analogues described herein can be used to treat a tumor, such as a breast cancer tumor, expressing β2-adrenergic receptor (AR), a cannabinoid (CB) receptor and epidermal growth factor receptor (EGFR).

Specifically it has been found that fenoterol analogues, such as (R,R 4′-methoxy-1-napthylfenoterol (MNF), inhibit the proliferation and growth of various types of breast cancer cells. In particular, a series of studies were performed to characterize fenoterol analogues and determine their possible therapeutic activities. MNF was observed to inhibit the growth of human-derived MCF-7 and MDA-MB-231 cancer cells which express all three of β2-adrenergic receptor (AR), a cannabinoid (CB) receptor and epidermal growth factor receptor (EGFR). Surprisingly MDA-MB-231 cancer cells were significantly more sensitive to MNF than were MCF-7 cancer cells. Thus, the compounds described herein can be used to treat breast cancer once the breast cancer has been screened to determine that it s a type that expresses all three of β2-adrenergic receptor (AR), a cannabinoid (CB) receptor and epidermal growth factor receptor (EGFR). Based upon these findings, disclosed are methods of treating disorders and diseases modulated by: i) CB receptor activity or expression (or both), such as GRP55 activity or expression (or both); ii) AR activity or expression (or both), such as (2-AR activity or expression (or both); and iii) EGFR activity or expression (or both).

### Abbreviations and Terms

### Abbreviations

AKAP: A-kinase anchoring protein
AM251: 1-(2,4-dichlorophenyl)-5-(4-iodophenyl)-4-methyl-N-(1-piperidyl)pyrazole-3-carboxamide
AM630: 1-[2-(morpholin-4-yl)ethyl]-2-methyl-3-(4-methoxybenzoyl)-6-iodoindole
AR: adrenergic receptor
2-AR: 2-adrenergic receptor
CB: cannabinoid
EGF: epidermal growth factor
EGFR: epidermal growth factor receptor
ERK: extracellular regulated kinase
Fen: fenoterol
GPR55: G protein-coupled receptor 55
GPCR: G protein-coupled receptor
HPLC: high performance liquid chromatography
IAM-PC: immobilized artificial membrane chromatographic support
ICI 118,551: 3-(isopropylamino)-1-[(7-methyl-4-indanyl)oxy]butan-2-ol
ICYP: [¹²⁵I]cyanopindolol
IP: intraperitoneal
IV: intravenous
MNF: 4-methoxy-1-naphthylfenoterol
NF: naphthylfenoterol
UV: ultraviolet

### Terms

Unless otherwise explained, all technical and scientific terms used herein have the same meaning as commonly understood by one of ordinary skill in the art to which the disclosed subject matter belongs. Definitions of common terms in chemistry may be found in The McGraw-Hill Dictionary of Chemical Terms, 1985, and The Condensed Chemical Dictionary, 1981.

Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description. Except as otherwise noted, the methods and techniques of the present invention are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

In order to facilitate review of the various embodiments disclosed herein, the following explanations of specific terms are provided:

Acyl: A group of the formula RC(O)— wherein R is an organic group.

Acyloxy: A group having the structure —OC(O)R, where R may be an optionally substituted alkyl or optionally substituted aryl. “Lower acyloxy” groups are those where R contains from 1 to 10 (such as from 1 to 6) carbon atoms.

Administration: To provide or give a subject a composition, such as a pharmaceutical composition including one or more fenoterol analogues by any effective route. Exemplary routes of administration include, but are not limited to, injection (such as subcutaneous, intramuscular, intradermal, intraperitoneal (IP), and intravenous (IV)), oral, sublingual, rectal, transdermal, intranasal, vaginal and inhalation routes.

Alkoxy: A radical (or substituent) having the structure —O—R, where R is a substituted or unsubstituted alkyl. Methoxy (—OCH₃) is an exemplary alkoxy group. In a substituted alkoxy, R is alkyl substituted with a non-interfering substituent. “Thioalkoxy” refers to —S—R, where R is substituted or unsubstituted alkyl. “Haloalkyloxy” means a radical —OR where R is a haloalkyl.

Alkoxy carbonyl: A group of the formula —C(O)OR, where R may be an optionally substituted alkyl or optionally substituted aryl. “Lower alkoxy carbonyl” groups are those where R contains from 1 to 10 (such as from 1 to 6) carbon atoms.

Alkyl: An acyclic, saturated, branched- or straight-chain hydrocarbon radical, which, unless expressly stated otherwise, contains from one to fifteen carbon atoms; for example, from one to ten, from one to six, or from one to four carbon atoms. This term includes, for example, groups such as methyl, ethyl, n-propyl, isopropyl, isobutyl, t-butyl, pentyl, heptyl, octyl, nonyl, decyl, or dodecyl. The term “lower alkyl” refers to an alkyl group containing from one to ten carbon atoms. Unless expressly referred to as an “unsubstituted alkyl,” alkyl groups can either be unsubstituted or substituted. An alkyl group can be substituted with one or more substituents (for example, up to two substituents for each methylene carbon in an alkyl chain). Exemplary alkyl substituents include, for instance, amino groups, amide, sulfonamide, halogen, cyano, carboxy, hydroxy, mercapto, trifluoromethyl, alkyl, alkoxy (such as methoxy), alkylthio, thioalkoxy, arylalkyl, heteroaryl, alkylamino, dialkylamino, alkylsulfano, keto, or other functionality.

Amino carbonyl (carbamoyl): A group of the formula C(O)N(R)R′, wherein R and R′ are independently of each other hydrogen or a lower alkyl group.

β2-adrenergic receptor ((2-AR): A subtype of adrenergic receptors that are members of the G-protein coupled receptor family. β2-AR subtype is involved in respiratory diseases, cardiovascular diseases, premature labor and, as disclosed herein, tumor development. Increased expression of β2-ARs can serve as therapeutic targets.

Cannabinoid Receptors: A class of cell membrane receptors under the G protein-coupled receptor superfamily. The cannabinoid receptors contain seven transmembrane spanning domains. Cannabinoid receptors are activated by three major groups of ligands, endocannabinoids (produced by the mammalian body), plant cannabinoids (such as THC, produced by the cannabis plant) and synthetic cannabinoids (such as HU-210). All of the endocannabinoids and plant cannabinoids are lipophilic, i.e., fat soluble, compounds. Two subtypes of cannabinoid receptors are CB₁ (see GenBank Accession No. NM_033181 mRNA and UniProt P21554, each of which is hereby incorporated by reference as of May 23, 2012) and CB₂ (see GenBank Accession No. NM_001841 mRNA and UniProt P34972, each of which is hereby incorporated by reference as of May 23, 2012). The CB₂ receptor is expressed mainly in the immune system and in hematopoietic cells. Additional non-CB₁ and non-CB₂ include GPR55 (GenBank Accession No. NM_005683.3 or NP_005674.2 protein, each of which is hereby incorporated by reference as of May 23, 2012), GPR119 (GenBank Accession No. NM_178471.2 or NP_848566.1 protein, each of which is hereby incorporated by reference as of May 23, 2012) and GPR18 (also known as N-arachidonyl glycine receptor and involved in microglial migration, GenBank Accession No. NM_001098200 mRNA, NP_001091670.1, each of which is hereby incorporated by reference as of May 23, 2012).

The protein sequences of CB₁ and CB₂ receptors are about 44% similar. When only the transmembrane regions of the receptors are considered, amino acid similarity between the two receptor subtypes is approximately 68%. In addition, minor variations in each receptor have been identified. Cannabinoids bind reversibly and stereo-selectively to the cannabinoid receptors. The affinity of an individual cannabinoid to each receptor determines the effect of that cannabinoid. Cannabinoids that bind more selectively to certain receptors are more desirable for medical usage. GPR55 is coupled to the G-protein G₁₃ and/or Gn and activation of the receptor leads to stimulation of rhoA, cdc42 and racl. GPR55 is activated by the plant cannabinoids A⁹-THC and cannabidiol, and the endocannabinoids anandamide, 2-AG, noladin ether in the low nanomolar range. In contrast, CB₁ and CB₂ receptors are coupled to inhibitory G proteins. This indicates that both types of receptors will have different readouts. For example, activation of CB₁ causes apoptosis whereas increase in GPR55 activity is oncogenic. The CB₁ receptor antagonist (also termed ‘inverse agonist’) compound, AM251, is, in fact, an agonist for GPR55. It binds GPR55 and is readily internalized. This illustrates the opposite behavior of these two GPCRs.

Carbamate: A group of the formula —OC(O)N(R)—, wherein R is H, or an aliphatic group, such as a lower alkyl group or an aralkyl group.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth. In one embodiment, a chemotherapeutic agent is an agent of use in treating neoplasms such as solid tumors, including a tumor associated with CB receptor activity and/or expression. In embodiments, a chemotherapeutic agent is radioactive molecule. In embodiments, a CB receptor regulator, such as one or more fenoterol analogues or a combination thereof is a chemotherapeutic agent. In one example, a chemotherapeutic agent is carmustine, lomustine, procarbazine, streptozocin, or a combination thereof. One of skill in the art can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L., Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Control or Reference Value: A “control” refers to a sample or standard used for comparison with a test sample. In some embodiments, the control is a sample obtained from a healthy subject or a tissue sample obtained from a patient diagnosed with a disorder or disease, such as a tumor, that did not respond to treatment with a p2-agonist. In some embodiments, the control is a historical control or standard reference value or range of values.

Derivative: A chemical substance that differs from another chemical substance by one or more functional groups. In embodiments, a derivative retains a biological activity of a molecule from which it was derived.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, an “effective amount” is a “therapeutically effective amount” in which the agent alone with an additional therapeutic agent(s) (for example a chemotherapeutic agent) induces the desired response such as treatment of a tumor. In one example, a desired response is to decrease tumor size or metastasis in a subject to whom the therapy is administered. Tumor metastasis does not need to be completely eliminated for the composition to be effective. For example, a composition can decrease metastasis by a desired amount, for example by at least 20%, at least 50%, at least 60%, at least 70%, at least 80%, at least 90%, at least 95%, at least 98%, or even at least 100% (elimination of the tumor), as compared to metastasis in the absence of the composition.

In particular examples, it is an amount of an agent effective to decrease a number of carcinoma cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

The effective amount of a composition useful for reducing, inhibiting, and/or treating a disorder in a subject will be dependent on the subject being treated, the severity of the disorder, and the manner of administration of the therapeutic composition. Effective amounts a therapeutic agent can be determined in many different ways, such as assaying for a reduction in tumor size or improvement of physiological condition of a subject having a tumor, such as a brain tumor. Effective amounts also can be determined through various in vitro, in vivo or in situ assays.

Fenoterol Analogues: “Fenoterol analogues” refers to (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol and their respective stereoisomers.

Inflammation: When damage to tissue occurs, the body's response to the damage is usually inflammation. The damage may be due to trauma, lack of blood supply, hemorrhage, autoimmune attack, transplanted exogenous tissue or infection. This generalized response by the body includes the release of many components of the immune system (for instance, IL-1 and TNF), attraction of cells to the site of the damage, swelling of tissue due to the release of fluid and other processes.

Isomers: Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that contain two or more chiral centers and are not mirror images of one another are termed “diastereomers.” Steroisomers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers, respectively). A chiral compound can exist as either an individual enantiomer or as a mixture thereof. A mixture containing equal proportions of the enantiomers is called a “racemic mixture.”

The compounds described herein may possess one or more asymmetric centers; such compounds can therefore be produced as individual (R), (S), (R,R′), (R,S′)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see, e.g., March, Advanced Organic Chemistry, 4th edition, New York: John Wiley and Sons, 1992, Chapter 4).

Optional: “Optional” or “optionally” means that the subsequently described event or circumstance can but need not occur, and that the description includes instances where said event or circumstance occurs and instances where it does not.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds or molecules, such as one or more nucleic acid molecules, proteins or antibodies that bind these proteins, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phenyl: Phenyl groups may be unsubstituted or substituted with one, two or three substituents, with substituent(s) independently selected from alkyl, heteroalkyl, aliphatic, heteroaliphatic, thioalkoxy, halo, haloalkyl (such as —CF₃), nitro, cyano, —OR (where R is hydrogen or alkyl), —N(R)R′ (where R and W are independently of each other hydrogen or alkyl), —COOR (where R is hydrogen or alkyl) or —C(O)N(R′)R″ (where W and R″ are independently selected from hydrogen or alkyl).

Purified: The term “purified” does not require absolute purity; rather, it is intended as a relative term. Thus, for example, a purified preparation is one in which a desired component such as an (R,R′)-enantiomer of fenoterol is more enriched than it was in a preceding environment such as in a (+)-fenoterol mixture. A desired component such as (R,R′)-enantiomer of fenoterol is considered to be purified, for example, when at least about 70%, 80%, 85%, 90%, 92%, 95%, 97%, 98%, or 99% of a sample by weight is composed of the desired component. Purity of a compound may be determined, for example, by high performance liquid chromatography (HPLC) or other conventional methods. In an example, the fenoterol analogue enantiomers are purified to represent greater than 90%, often greater than 95% of the other enantiomers present in a purified preparation. In other cases, the purified preparation may be essentially homogeneous, wherein other stereoisomers are less than 1%.

Compounds described herein may be obtained in a purified form or purified by any of the means known in the art, including silica gel and/or alumina chromatography. See, e.g., Introduction to Modern Liquid Chromatography, 2nd Edition, ed. by Snyder and Kirkland, New York: John Wiley and Sons, 1979; and Thin Layer Chromatography, ed. by Stahl, New York: Springer Verlag, 1969. In an example, a compound includes purified fenoterol or fenoterol analogue with a purity of at least about 70%, 80%, 85%, 90%, 92%, 95%, 97%, 98%, or 99% of a sample by weight relative to other contaminants. In a further example, a compound includes at least two purified stereoisomers each with a purity of at least about 70%, 80%, 85%, 90%, 92%, 95%, 97%, 98%, or 99% of a sample by weight relative to other contaminants. For instance, a compound can include a substantially purified (R,R′)-fenoterol analogue and a substantially purified (R,S′)-fenoterol analogue.

Subject: The term “subject” includes both human and veterinary subjects, for example, humans, non-human primates, dogs, cats, horses, rats, mice, and cows. Similarly, the term mammal includes both human and non-human mammals.

Tissue: A plurality of functionally related cells. A tissue can be a suspension, a semi-solid, or solid. Tissue includes cells collected from a subject such as the brain or a portion thereof.

Tumor: All neoplastic cell growth and proliferation, whether malignant or benign, and all pre-cancerous and cancerous cells and tissues. A primary tumor is tumor growing at the anatomical site where tumor progression began and proceeded to yield this mass.

Under conditions sufficient for: A phrase that is used to describe any environment that permits the desired activity. In one example, under conditions sufficient for includes administering one or more fenoterol analogues, fenoterol or a combination thereof to a subject to at a concentration sufficient to allow the desired activity. In some examples, the desired activity is reducing or inhibiting a sign or symptom associated with a disorder or disease, such as a breast cancer, can be evidenced, for example, by a delayed onset of clinical symptoms of the tumor in a susceptible subject, a reduction in severity of some or all clinical symptoms of the tumor, a slower progression of the tumor (for example by prolonging the life of a subject having the tumor), a reduction in the number of tumor reoccurrence, an improvement in the overall health or well-being of the subject, or by other parameters well known in the art that are specific to the particular disease. In one particulate example, the desired activity is preventing or inhibiting tumor growth, such as breast cancer growth. Tumor growth does not need to be completely inhibited for the treatment to be considered effective. For example, a partial reduction or slowing of growth such as at least about a 10% reduction, such as at least 20%, at least about 30%, at least about 40%, at least about 50% or greater is considered to be effective.

### Chemical Structure of Fenoterol Analogues

Exemplary fenoterol analogues useful in the methods of the present disclosure include (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol and their respective stereoisomers.

Examples of suitable groups for R₁-R₃ that can be cleaved in vivo to provide a hydroxy group include, without limitation, acyl, acyloxy and alkoxy carbonyl groups. Compounds having such cleavable groups are referred to as “prodrugs.” The term “prodrug,” as used herein, means a compound that includes a substituent that is convertible in vivo (e.g., by hydrolysis) to a hydroxyl group. Various forms of prodrugs are known in the art, for example, as discussed in Bundgaard, (ed.), Design of Prodrugs, Elsevier (1985); Widder, et al. (ed.), Methods in Enzymology, Vol. 4, Academic Press (1985); Krogsgaard-Larsen, et al., (ed), Design and Application of Prodrugs, Textbook of Drug Design and Development, Chapter 5, 113 191 (1991); Bundgaard, et al., Journal of Drug Delivery Reviews, 8: 1 38 (1992); Bundgaard, Pharmaceutical Sciences, 77:285 et seq. (1988); and Higuchi and Stella (eds.) Prodrugs as Novel Drug Delivery Systems, American Chemical Society (1975).

In embodiments, administering includes administering a therapeutically effective amount of a fentoerol analogue. In embodiments, administering includes administering a therapeutically effective amount of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol, their respective stereoisomers, or a combination thereof. In embodiments, administering comprises administering a therapeutically effective amount of:

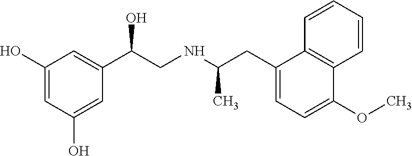 [SMILES: COc1ccc(C[C@@H](C)NC[C@H](O)c2cc(O)cc(O)c2)c2ccccc12]

Particular method embodiments contemplate the use of solvates (such as hydrates), pharmaceutically acceptable salts and/or different physical forms of the fenoterol analogues herein described.

### Solvates, Salts and Physical Forms

“Solvate” means a physical association of a compound with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol-associated compound, methanol-associated compounds, and the like. “Hydrate” is a solvate wherein the solvent molecule(s) is/are H₂O.

The disclosed compounds also encompass salts including, if several salt-forming groups are present, mixed salts and/or internal salts. The salts are generally pharmaceutically acceptable salts that are non-toxic. Salts may be of any type (both organic and inorganic), such as fumarates, hydrobromides, hydrochlorides, sulfates and phosphates. In an example, salts include non-metals (e.g., halogens) that form group VII in the periodic table of elements. For example, compounds may be provided as a hydrobromide salt.

Additional examples of salt-forming groups include, but are not limited to, a carboxyl group, a phosphonic acid group or a boronic acid group, that can form salts with suitable bases. These salts can include, for example, nontoxic metal cations, which are derived from metals of groups IA, IB, IIA and IIB of the periodic table of the elements. In one embodiment, alkali metal cations such as lithium, sodium or potassium ions, or alkaline earth metal cations such as magnesium or calcium ions can be used. The salt can also be a zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2-hydroxyethyl)amine, or N-methyl-D-glucamine, or quaternary ammonium compounds such as tetrabutylammonium salts.

Exemplary compounds disclosed herein possess at least one basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with a-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In a currently preferred embodiment, fenoterol is provided as a hydrobromide salt and exemplary fenoterol analogues are provided as their fumarate salts.

Additional counterions for forming pharmaceutically acceptable salts are found in Remington's Pharmaceutical Sciences, 19th Edition, Mack Publishing Company, Easton, Pa., 1995. In one aspect, employing a pharmaceutically acceptable salt may also serve to adjust the osmotic pressure of a composition.

In certain embodiments the compounds used in the method are provided are polymorphous. As such, the compounds can be provided in two or more physical forms, such as different crystal forms, crystalline, liquid crystalline or non-crystalline (amorphous) forms.

### Use for the Manufacture of a Medicament

Any of the above described compounds (e.g., (R,R′) and/or (R,S′) fenoterol analogues or a hydrate or pharmaceutically acceptable salt thereof) or combinations thereof are intended for use in the manufacture of a medicament for treatment of breast cancer.

Formulations suitable for such medicaments, subjects who may benefit from same and other related features are described elsewhere herein.

### Methods of Synthesis

The disclosed fenoterol analogues can be synthesized by any method known in the art including those described in U.S. Patent Application Publication No. US 2010-0168245 A1, U.S. Patent Application Publication No. US 2012-0157543 A1 and International Patent Publication No. WO 2011/112867, each of which is hereby incorporated by reference in its entirety. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein may be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via open column chromatography or prep chromatography.

Suitable exemplary syntheses of fenoterol analogues are provided below:

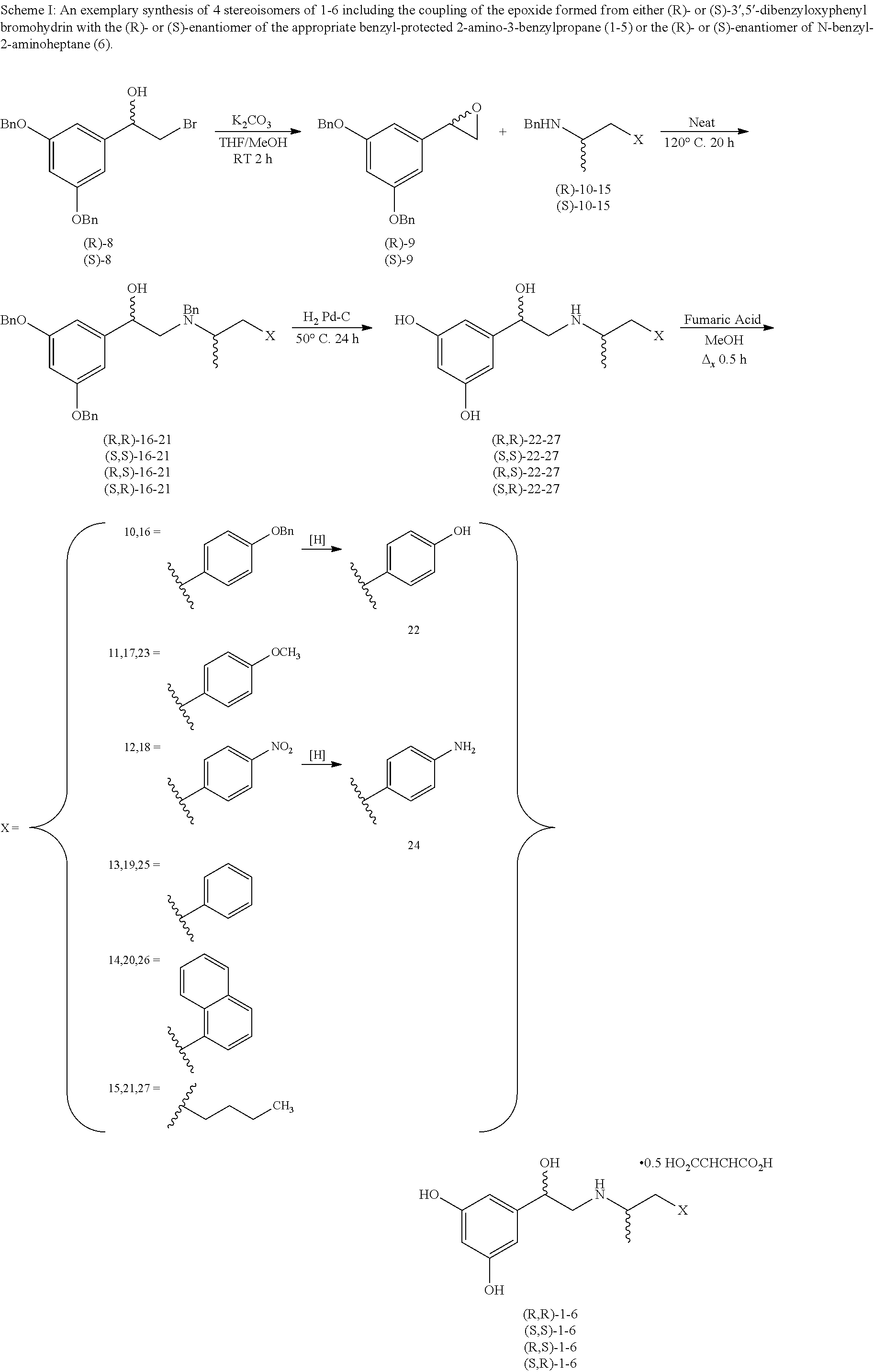

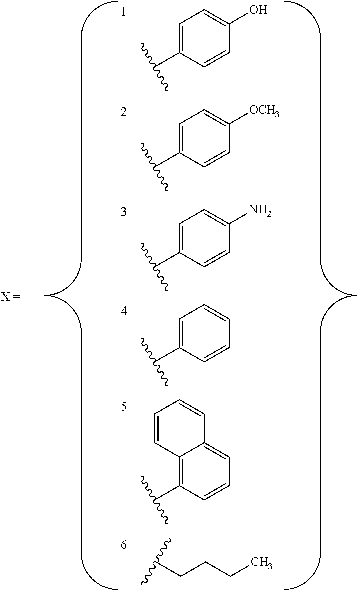

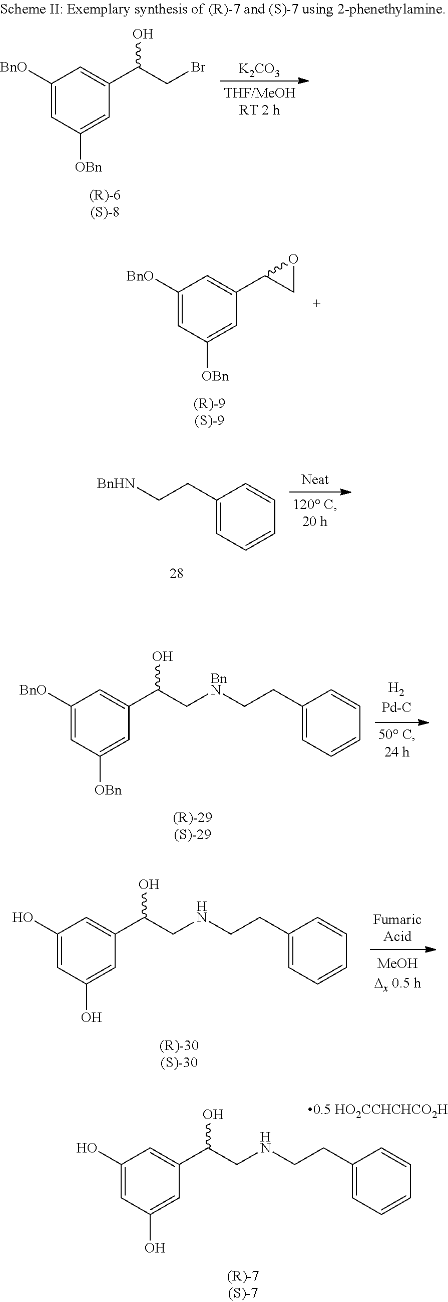

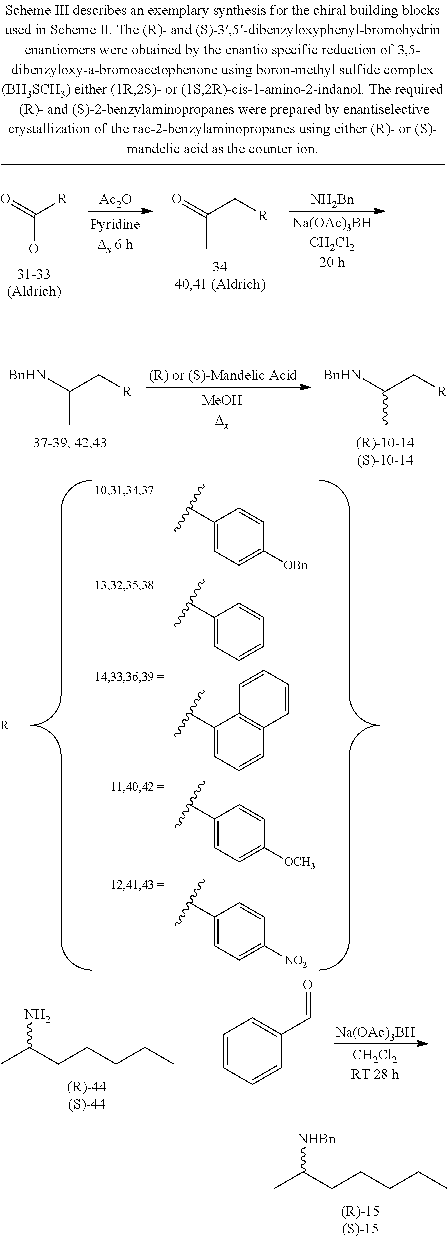

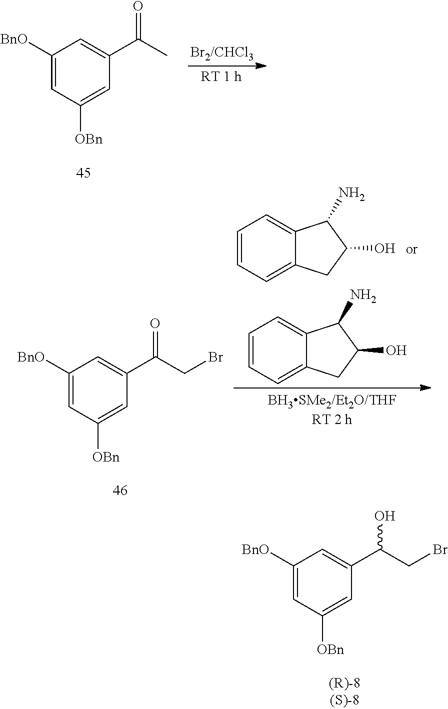

### Pharmaceutical Compositions

The disclosed fenoterol analogues can be useful, at least, for reducing or inhibiting one or more symptoms or signs associated with breast cancer. Accordingly, pharmaceutical compositions comprising at least one disclosed fenoterol analogue are also described herein.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of (R,R′)-fenoterol and disclosed fenoterol analogues. Pharmaceutical compositions comprising at least one of these compounds can be formulated for use in human or veterinary medicine. Particular formulations of a disclosed pharmaceutical composition may depend, for example, on the mode of administration (e.g., oral or parenteral) and/or on the disorder to be treated. In some embodiments, formulations include a pharmaceutically acceptable carrier in addition to at least one active ingredient, such as a fenoterol compound.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle.

For solid compositions such as powder, pill, tablet, or capsule forms conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances or excipients, such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

The disclosed pharmaceutical compositions may be formulated as a pharmaceutically acceptable salt. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydriodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 19th Edition, Mack Publishing Company, Easton, Pa., 1995. A pharmaceutically acceptable salt may also serve to adjust the osmotic pressure of the composition.

The dosage form of a disclosed pharmaceutical composition will be determined by the mode of administration chosen. For example, in addition to injectable fluids, oral dosage forms may be employed. Oral formulations may be liquid such as syrups, solutions or suspensions or solid such as powders, pills, tablets, or capsules. Methods of preparing such dosage forms are known, or will be apparent, to those skilled in the art.

Certain embodiments of the pharmaceutical compositions comprising a disclosed compound may be formulated in unit dosage form suitable for individual administration of precise dosages. The amount of active ingredient such as (R,R′)-MNF or NF administered will depend on the subject being treated, the severity of the disorder, and the manner of administration, and is known to those skilled in the art. Within these bounds, the formulation to be administered will contain a quantity of the extracts or compounds disclosed herein in an amount effective to achieve the desired effect in the subject being treated.

In particular examples, for oral administration the compositions are provided in the form of a tablet containing from about 1.0 to about 50 mg of the active ingredient, particularly about 2.0 mg, about 2.5 mg, 5 mg, about 10 mg, or about 50 mg of the active ingredient for the symptomatic adjustment of the dosage to the subject being treated. In one exemplary oral dosage regimen, a tablet containing from about 1 mg to about 50 mg (such as about 2 mg to about 10 mg) active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

In embodiments, the pharmaceutical composition is administered without concurrent administration of a second agent for the treatment of breast cancer. In one specific, non-limiting example, one or more of the disclosed compositions is administered without concurrent administration of other agents, such as without concurrent administration of an additional agent also known to target the tumor. In other specific non-limiting examples, a therapeutically effective amount of a disclosed pharmaceutical composition is administered concurrently with an additional agent, including an additional therapy. For example, the disclosed compounds are administered in combination with a chemotherapeutic agent, anti-oxidants, anti-inflammatory drugs or combinations thereof.

In other examples, a disclosed pharmaceutical composition is administered as adjuvant therapy. For example, a pharmaceutical composition containing one or more of the disclosed compounds is administered orally daily to a subject in order to prevent or retard tumor growth. In one particular example, a composition containing equal portions of two or more disclosed compounds is provided to a subject. In one example, a composition containing unequal portions of two or more disclosed compounds is provided to the subject. For example, a composition contains unequal portions of a (R,R′)-fenoterol derivative and a (S,R′)-fenoterol derivative and/or a (R,S′)-derivative. In one particular example, the composition includes a greater amount of the (S,R′)- or (R,S′)-fenoterol derivative. Such therapy can be given to a subject for an indefinite period of time to inhibit, prevent, or reduce tumor reoccurrence.

### Methods of Use

The present disclosure includes methods of treating disorders including reducing or inhibiting one or more signs or symptoms associated with breast cancer. In some examples, the tumor is a primary tumor. In some examples, the tumor is a breast cancer expressing β2-adrenergic receptor (AR), cannabinoid (CB) receptor and epidermal growth factor receptor (EGFR). The fenoterol analogue and/or fenoterol, such as (R,R′) fenoterol, itself is administered depending upon the tumor receptor population.

Disclosed methods include administering a fenoterol analogue (and, optionally, one or more other pharmaceutical agents) depending upon the receptor population of the tumor, to a subject in a pharmaceutically acceptable carrier and in an amount effective to treat the tumor expressing a combination of β 2-AR, a CB receptor and EGFR. Treatment of a tumor includes preventing or reducing signs or symptoms associated with the presence of such tumor (for example, by reducing the size or volume of the tumor or a metastasis thereof). Such reduced growth can in some examples decrease or slow metastasis of the tumor, or reduce the size or volume of the tumor by at least 10%, at least 20%, at least 50%, or at least 75%, such as between 10%-90%, 20%-80%, 30%-70%, 40%-60%, including a 10%, 15%, 20%, 25%, 30%, 35%, 40%, 45%, 50%, 55%, 60%, 65%, 70%, 75%, 80%, 90%, or 95% reduction. In another example, treatment includes reducing the invasive activity of the tumor in the subject, for example by reducing the ability of the tumor to metastasize. In some examples, treatment using the methods disclosed herein prolongs the time of survival of the subject.

Routes of administration useful in the disclosed methods include but are not limited to oral and parenteral routes, such as intravenous (IV), intraperitoneal (IP), rectal, topical, ophthalmic, nasal, and transdermal as described in detail above.

An effective amount of a fenoterol analogue will depend, at least, on the particular method of use, the subject being treated, the severity of the tumor, and the manner of administration of the therapeutic composition.

A “therapeutically effective amount” of a composition is a quantity of a specified compound sufficient to achieve a desired effect in a subject being treated. For example, this may be the amount of a fenoterol analogue necessary to prevent or inhibit tumor growth and/or one or more symptoms associated with the tumor in a subject. Ideally, a therapeutically effective amount of a fenoterol analogue is an amount sufficient to prevent or inhibit tumor growth and/or one or more symptoms associated with the tumor in a subject without causing a substantial cytotoxic effect on host cells.

Therapeutically effective doses of a disclosed fenoterol compound or pharmaceutical composition can be determined by one of skill in the art, with a goal of achieving concentrations that are at least as high as the IC₅₀ of the applicable compound disclosed in the examples herein. An example of a dosage range is from about 0.001 to about 10 mg/kg body weight orally in single or divided doses. In particular examples, a dosage range is from about 0.005 to about 5 mg/kg body weight orally in single or divided doses (assuming an average body weight of approximately 70 kg; values adjusted accordingly for persons weighing more or less than average). For oral administration, the compositions are, for example, provided in the form of a tablet containing from about 1.0 to about 50 mg of the active ingredient, particularly about 2.5 mg, about 5 mg, about 10 mg, or about 50 mg of the active ingredient for the symptomatic adjustment of the dosage to the subject being treated. In one exemplary oral dosage regimen, a tablet containing from about 1 mg to about 50 mg active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

The specific dose level and frequency of dosage for any particular subject may be varied and will depend upon a variety of factors, including the activity of the specific compound, the metabolic stability and length of action of that compound, the age, body weight, general health, sex and diet of the subject, mode and time of administration, rate of excretion, drug combination, and severity of the condition of the subject undergoing therapy.

### Selecting a Subject

Subjects can be screened prior to initiating the disclosed therapies, for example to select a subject in need of or at risk of developing a disorder or disease regulated by β2-adrenergic receptor (AR) activity or expression, cannabinoid (CB) receptor activity or expression, and epidermal growth factor receptor (EGFR) activity or expression. Briefly, the method can include screening subjects to determine if they have or are at risk of developing such a disorder or disease, such as if the subject is in need of breast cancer inhibition. Subjects having a tumor that expresses β2-adrenergic receptor (AR), cannabinoid (CB) receptor, and epidermal growth factor receptor (EGFR) or at risk of developing such a tumor are selected. In one example, subjects are diagnosed with the tumor by clinical signs, laboratory tests, or both.

In an example, a subject in need of the disclosed therapies is selected by detecting a tumor expressing β2-adrenergic receptor (AR), cannabinoid (CB) receptor, and epidermal growth factor receptor (EGFR) or regulated by their activity, such as by detecting β2-adrenergic receptor (AR) activity, cannabinoid (CB) receptor activity, and epidermal growth factor receptor (EGFR) activity or expression in a sample obtained from a subject identified as having, suspected of having or at risk of acquiring such a tumor. For example, detection of altered, such as at least a 10% alteration, including a 10%-90%, 20%-80%, 30%-70%, 40%-60%, such as a 10%, 15%, 20%, 25%, 30%, 35%, 40%, 45%, 50%, 55%, 60%, 65%, 70%, 75%, 80%, 90%, 95% alteration or more in β2-adrenergic receptor (AR) expression or activity, cannabinoid (CB) receptor expression or activity, and epidermal growth factor receptor (EGFR) expression or activity as compared to β2-adrenergic receptor (AR) expression or activity, cannabinoid (CB) receptor expression or activity, and epidermal growth factor receptor (EGFR) expression or activity in the absence of a primary tumor, indicates that the tumor can be treated using the fenoterol compositions and methods provided herein.

Pre-screening is not required prior to administration of the therapeutic agents disclosed herein (such as those including fenoterol, a fenoterol analogue or a combination thereof).

### Assessment

Following the administration of one or more therapies, subjects having a disorder or disease regulated by β2-adrenergic receptor (AR) expression or activity, cannabinoid (CB) receptor expression or activity, and epidermal growth factor receptor (EGFR) expression or activity can be monitored for decreases in tumor growth, tumor volume or in one or more clinical symptoms associated with the tumor. In particular examples, subjects are analyzed one or more times, starting 7 days following treatment. Subjects can be monitored using any method known in the art including those described herein including imaging analysis.

### Additional Treatments and Additional Therapeutic Agents

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least a 20%, at least a 30%, at least a 40%, at least a 50%, or at least a 70% reduction in one or more signs or symptoms associated with the disorder or disease, such as a tumor regulated by β2-adrenergic receptor (AR) expression or activity, cannabinoid (CB) receptor expression or activity, and epidermal growth factor receptor (EGFR) expression or activity, including tumor size or volume.

In some examples, the method further includes administering a therapeutic effective amount of a fenoterol analogue with additional therapeutic treatments. In particular examples, prior to, during, or following administration of a therapeutic amount of an agent that prevents or inhibits a tumor regulated by β2-adrenergic receptor (AR) expression or activity, cannabinoid (CB) receptor expression or activity, and epidermal growth factor receptor (EGFR) expression or activity, the subject can receive one or more other therapies. In one example, the subject receives one or more treatments to remove or reduce the tumor prior to administration of a therapeutic amount of a composition including fenoterol, a fenoterol analogue or combination thereof.

Examples of such therapies include, but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. Particular examples of additional therapeutic agents that can be used include microtubule-binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

“Microtubule-binding agent” refers to an agent that interacts with tubulin to stabilize or destabilize microtubule formation thereby inhibiting cell division. Examples of microtubule-binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds are of use in the methods described herein: Suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Examples of alkylating agents include carmustine or lomustine. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevac, Iressa, and Tarceva that prevent phosphorylation and activation of growth factors.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogues thereof.

In one example, at least a portion of the tumor is surgically removed (for example via cryotherapy), irradiated, chemically treated (for example via chemoembolization) or combinations thereof, prior to administration of the disclosed therapies (such as administration of a fenoterol analogue). For example, a subject having a breast cancer associated with β2-adrenergic receptor (AR) expression or activity, cannabinoid (CB) receptor expression or activity, and epidermal growth factor receptor (EGFR) expression or activity can have at least a portion of the tumor surgically excised prior to administration of the disclosed therapies. In an example, one or more chemotherapeutic agents are administered following treatment with a composition including a fenoterol analogue.

The subject matter of the present disclosure is further illustrated by the following non-limiting Examples.

### Materials and Methods

The material and methods used for Examples 2-4 were as follows:

Materials.

(R,R′)-, (R,S′)-, (S,R)- and (S,S′)-fenoterol and the fenoterol analogs, (R,R′)-ethylfenoterol, (R,R′) 4′-aminofenoterol, (R,R′)-1-naphthylfenoterol and (R,R′)- and (R,S′)-4-methoxy-1-naphthylfenoterol, were synthesized as previously described (Jozwiak et ah, J Med Chem 50:2903-2915, 2007; Jozwiak et al, Bioorg Med Chem 18:728-736, 2010; each of which is incorporated by reference in its entirety). [³H]-Thymidine (70-90 Ci/mmol) was purchased from PerkinElmer Life and Analytical Sciences (Waltham, Mass.). Eagle's Minimum Essential Medium (E-MEM), trypsin solution, phosphate-buffered saline (PBS), fetal bovine serum (FBS), 100× solutions of sodium pyruvate (100 mM), L-glutamine (200 mM), and penicillin/streptomycin (a mixture of 10,000 units/ml penicillin and 10,000 μg/ml streptomycin) were obtained from Quality Biological (Gaithersburg, Md.). WIN 55,212-2, AM251, and AM630 were purchased from Cayman Chemical (Ann Arbor, Mich.). ICI 118,551 hydrochloride and (R)-isoproterenol were obtained from Sigma-Aldrich (St. Louis, Mo.). Phenylmethylsulfonyl fluoride (PMSF), benzamidine, leupeptin, pepstatin A, MgCl₂, EDTA, Trizma-Hydrochloride (Tris-HCl), (±)-propranolol and minimal essential medium (MEM) were obtained from Sigma Aldrich (St. Louis, Mo.). Egg phosphatidylcholine lipids (PC) were obtained from Avanti Polar Lipids (Alabaster, Ala.). (±)-fenoterol was purchased from Sigma Aldrich and [³H]-(±)-fenoterol was acquired from Amersham Biosciences (Boston, Mass.). The organic solvents n-hexane, 2-propanol and triethylamine were obtained as ultra pure HPLC grade solvents from Carlo Erba (Milan, Italy). Fetal bovine serum and penicillin-streptomycin were purchased from Life Technologies (Gaithersburg, Md.), and [¹²⁵I]-(i)-iodocyanopindolol (ICYP) was purchased from NEN Life Science Products, Inc. (Boston, Mass.).

Maintenance and Treatment of Cell Lines.

Human MCF-land MDA-MD-231 breast cancer cells (ATCC, Manassas, Va.) were cultured in RPMI-1640 supplemented with 10% fetal bovine serum, 4 mM L-glutamine, 100 U/ml penicillin and 0.1 mg/ml streptomycin.

[³H]-Thymidine Incorporation Assay.

Cells were seeded in 12-well plates at approximately 50,000 cells/well and incubated at 37° C. After 24 hours, the wells were rinsed with PBS and replaced with serum-free medium containing the appropriate concentration of the test compounds. After another 24-hour incubation at 37° C., 1 μCi of [³H]-thymidine was added to each well and incubated at 37° C. for 16 hours. [³H]-Thymidine incorporation into DNA was monitored after the cells were washed twice with PBS and then lysed in 600 μl of 0.1 N NaOH for 30 minutes with shaking. The lysate was then mixed with 3 mL of liquid scintillation cocktail (Beckman Coulter, Inc., Brea, Calif.), and radioactivity was measured by liquid scintillation counting using Beckman Coulter LS6000IC Scintillation Counter. Data are shown as CPM incorporated compared to the control cells.

[³⁵S] Methionine Labeling.

Serum starved cells were incubated in serum-free medium without methionine and cysteine and treated with (R,R′)-MNF. Then, [³⁵S]Met/Cys labeling was carried out. Cell lysates were resolved by SDS-polyacrylamide gel electrophoresis and transferred onto PVD membrane, and autoradiography was performed. The membrane was probed with anti-β-actin antibody to confirm equal protein loading.

Wound Healing Assay.

A scratch wound was made with a cell scraper. Cells were pictured using Axiovert 200 microscope and AxioCam HRc camera. (Carl Zeiss)

ELISA.

Levels of ERK1/2 and its phosphorylated forms were determined using PathScan ELISA kits according to manufacturer's protocol (Perkin Elmer Alphascreen Surefire ERK1/2 assay kit, cat no. TGRES500).

Migration and Invasion.

Assays were performed using xCELLigence RTCA Analyzer. Serum-starved cells were allowed to migrate or invade towards 10% FBS via bare microporous PET membrane or Matrigel-coated membrane, respectively.

Western Blotting.

Cells were lysed with RIPA buffer containing EGTA and EDTA (Boston BioProducts, Ashland, Mass.). The lysis buffer was mixed with a protease inhibitor cocktail (Sigma-Aldrich). Protein concentrations were measured using the bicinchoninic acid reagent (Thermo-Pierce Biotechnology, Inc., Rockford, Ill.). Proteins (20 μg/well) were separated on 4-12% precast gels (Invitrogen, Carlsbad, Calif.) using SDS-polyacrylamide gel electrophoresis under reducing conditions and were electrophoretically transferred onto polyvinylidene fluoride membrane (Invitrogen). Western blots were performed according to standard methods. The visualization of immunoreactive bands was performed using the ECL Plus Western Blotting Detection System (GE Healthcare, NJ) and their quantification was done by volume densitometry using Image J software and normalization to β-actin. The primary antibody for β2-APν was obtained from Enzo Life Sciences, Inc. (Cat. # AD 1-905-742-100, Farmingdale, N.Y.); rabbit anti-phospho-Akt (Ser-473), phospho-ERK1/2, total Akt and total ERK2 were from Cell Signaling Technology (Beverly, Mass.), and anti-P-actin was from Abeam (Cambridge, Mass.). The antibodies were used at a dilution recommended by the manufacturer.

Statistical Analysis.

Results were expressed as relative to the control value. Studies were performed in at least two to three different culture preparations, and two to three dishes for each test condition were plated in each preparation. Results are expressed as means+S.E. Student's t-test was used to make statistical comparisons between groups. Analyses were performed using the SigmaPlot Software (Systat Software, Inc. San Jose, Calif.), Graphpad Prism 4 (GraphPad Software, Inc., La Jolla, Calif.) and Microsoft® Office Excel, 2003 (Microsoft Corp., Redmond, Wash.), with p values <0.05 considered significant.

cAMP Accumulation.

HepG2 cells were seeded in 96-well plates and grown to confluency. Cells were rinsed in Krebs-HEPES buffer, pH 7.4, pre-incubated for 10 minutes with the buffer, and then 10 μM (R)-isoproterenol or (R,R′)-fenoterol was added followed by incubation for an additional 10 minutes. The levels of cAMP accumulated in cells were determined and normalized to the amount of protein per well.

RNA Extraction, cDNA Synthesis, and RT-PCR Analysis. Total RNA was isolated from HepG2, 1321N1 and U87MG cells using the RNeasy Mini kit (Qiagen, Valencia, Calif.). The RNA preparation included a DNAse digestion step. RNA concentration and quality was measured using the NanoDrop spectrophotometer (NanoDrop Technologies, Wilmington, Del.). To obtain cDNA, 1 μg total RNA was reverse-transcribed using the Promega reverse transcription kit (Promega Corp., Madison, Wis.). PCR reactions were performed to determine the expression of CB1R, CB2R, and 2-AR mRNAs using GAPDH as internal control. The PCR primers and conditions are found in Supplemental Table 1.

Cell Cycle Analysis.

Cell cycle distributions were performed by flow cytometry on propidium iodide-stained nuclei prepared by the NEVI technique. DNA histograms of at least 10,000 cells acquired on a Becton-Dickinson FACScanto II (BD Biosciences, San Jose, Calif.) were deconvoluted using the Multicycle program (Phoenix Flow Systems) for estimates of the percentage of cells in the G0/1, S, and G2+M phases of the cell cycle. Debris and doublets were removed from the analysis by software algorithms.

Apoptosis Assay.

The degree of apoptosis induced by drug treatment was assayed by flow cytometry using the Alexa Fluor® 488 annexin V/Dead Cell Apoptosis Kit (Invitrogen) following the standard manufacturer's protocol. Briefly, HepG2 cells (5×10⁵) were grown on 100-mm dishes for 24 hours followed by treatment with vehicle, (R,R′)-fenoterol, or (R,R′)-MNF, all in serum-free medium. Cells were subsequently harvested after a 24-hour incubation, washed in cold PBS, and resuspended in 100 μL of IX annexin-binding buffer to maintain a density ˜1×10⁶ cells/mL, after which 5 μl Alexa Fluor® 488 annexin V and 1 100 μg/mL propidium iodide were added to the cell suspensions. Cells were then incubated at room temperature for 15 minutes and 400 μl IX annexin-binding buffer was added followed by gentle mixing. Stained cells were analyzed on a BDFACSCanto II flow cytometer.

### Example 1

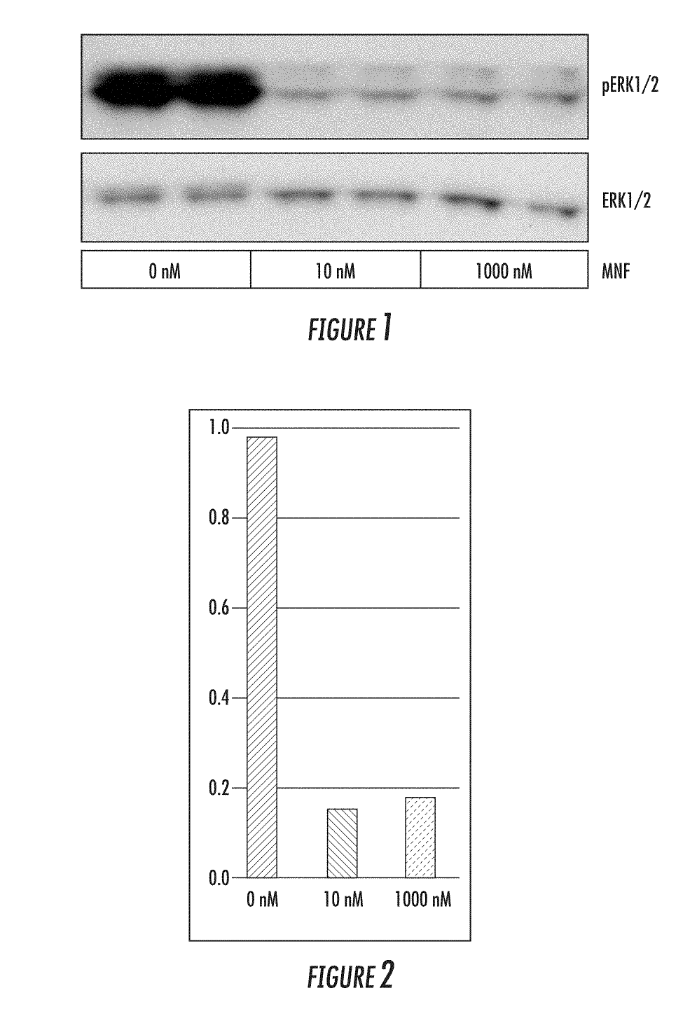

(R,R′)-MNF decreased basal phosphorylation levels of ERK. MCF-7 and MDA-MB-231 cells were serum starved for 3 hours and subjected to compound of interest or vehicle for 15 minutes. The levels of phosphorylated ERK were determined in MNF-treated MCF-7 and MDA-MB-231 cells. The results are shown in FIGS. 1 and 2.

### Example 2

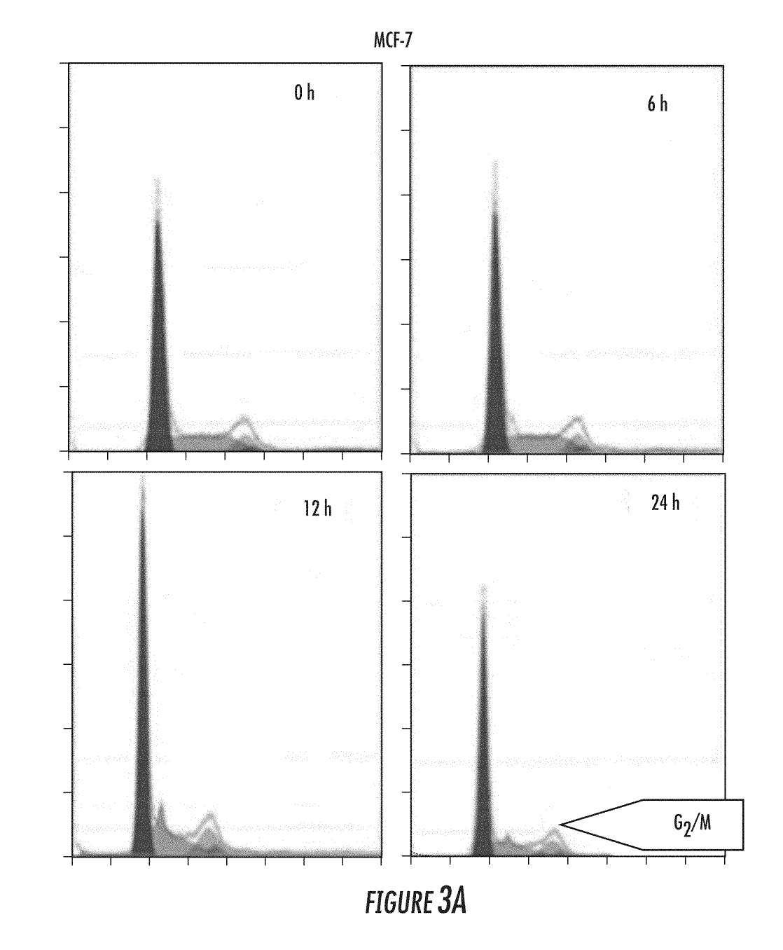

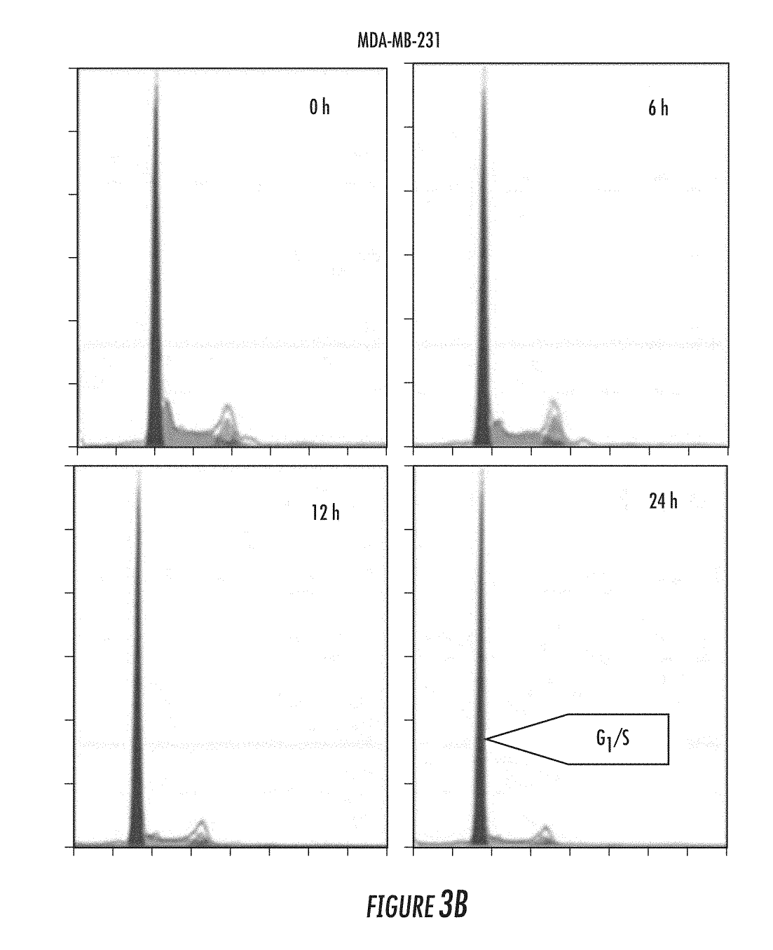

Cell cycle analysis revealed that MNF causes arrest of the cell cycle in MCF-7 and MDA-MB-231 cells. G2/M phase arrested cell population was increased in MCF-7 cells and MDA-MB-231 cells were arrested at G1/S phase. The DNA histograms generated are shown in FIGS. 3A and 3B.

### Example 3

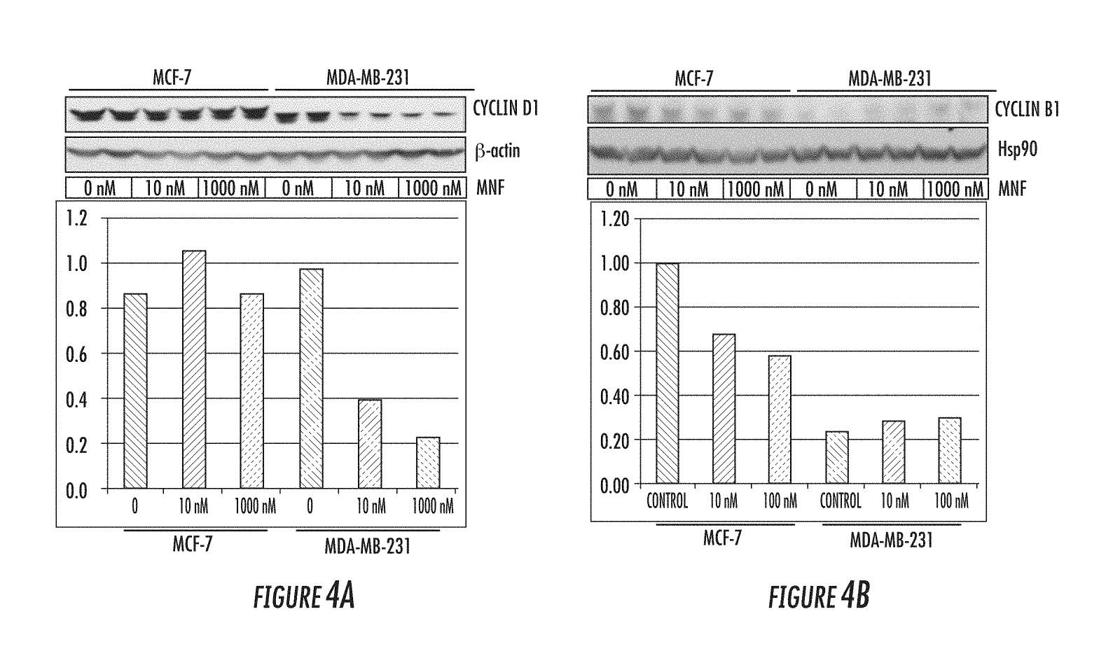

The effect of MNF on expression of the cell cycle regulatory proteins Cyclin D1 and Cyclin B1 in MCF-7 cells and MDA-MB-231 cells was evaluated. Expression of Cyclin D1 was reduced in MDA-MB-231 cells, whereas unchanged in MCF-7 cells. In contrast, MNF reduced expression of Cyclin B1 in MCF-7 cells. These results are shown in FIGS. 4A and 4B.

### Example 4

The effect of MNF on the expression of Multidrug resistance protein (Mdr) in MCF-7 cells was tested using techniques know to those skilled in the art. The results of these experiments are shown in FIG. 5. As shown therein, MNF reduces the expression of Multidrug resistance protein (Mdr) in MCF-7 cells.

### Example 5

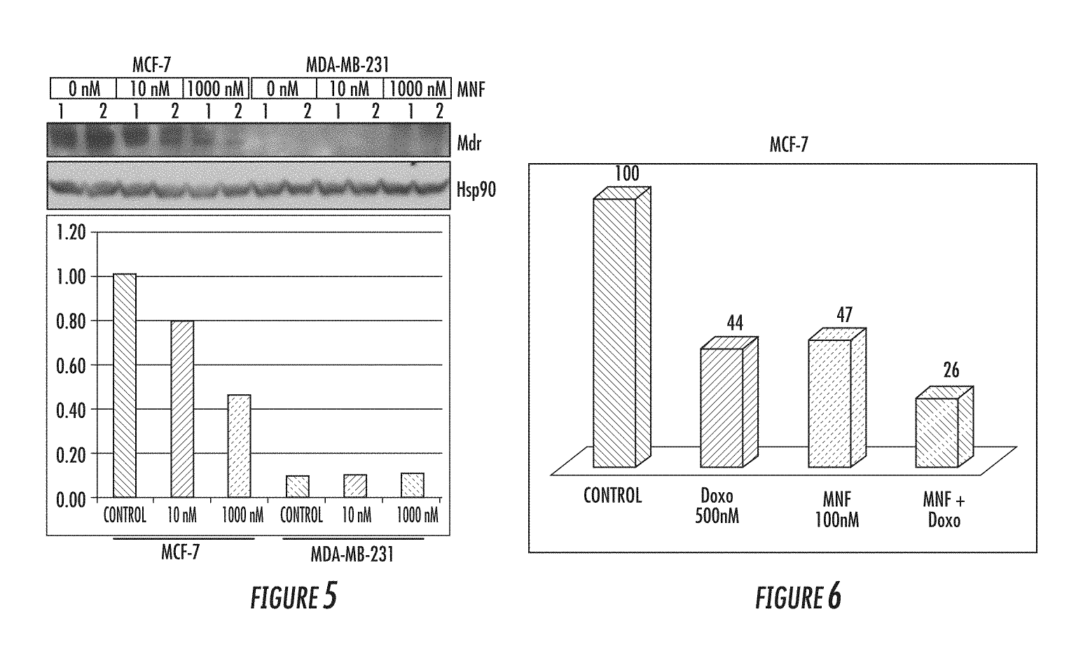

The anti-proliferative effect of MNF, Doxorubicin and a combination of MNF and Doxorubicin on MCF-7 cells was tested using techniques know to those skilled in the art. The results of these experiments are shown in FIG. 6. As shown therein, the combination of Doxorubicin and MNF showed enhanced the anti-proliferative properties in MCF-7 cells compared to MNF alone or Doxorubicin alone.

### Example 6

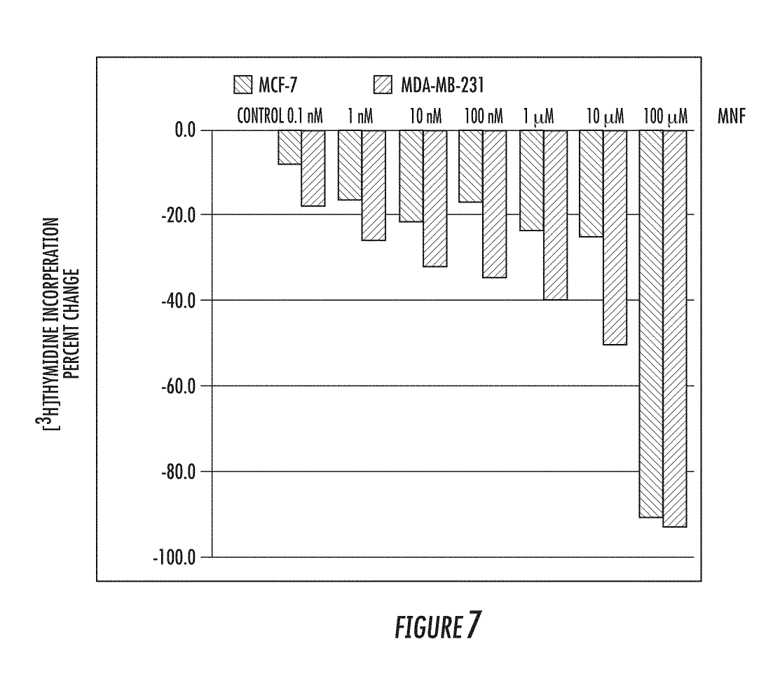

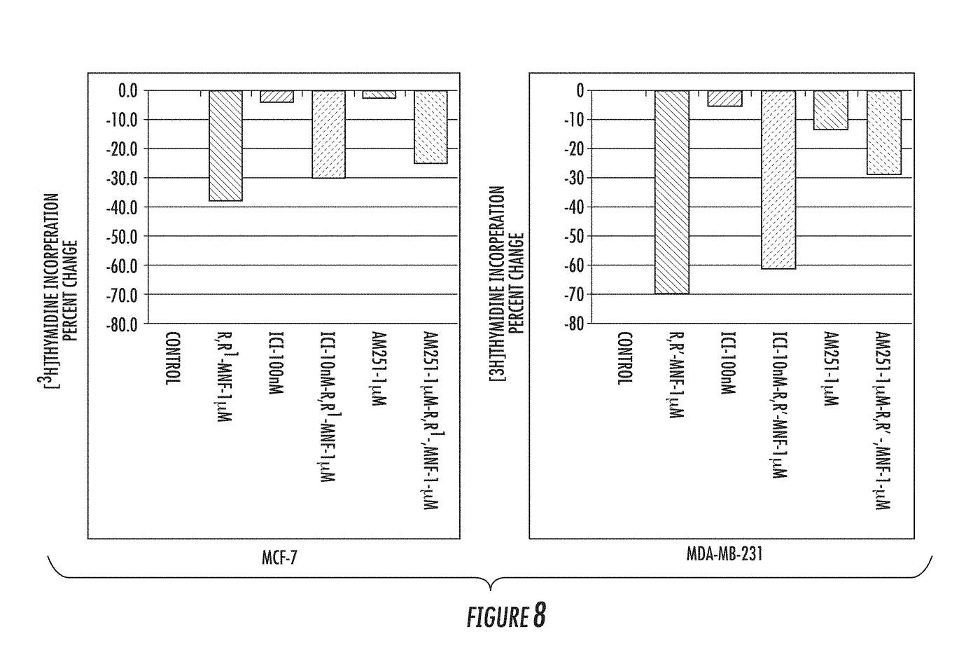

(R,R′)-MNF was found to inhibit proliferation of MCF-7 cells and MDA-MB-231 cells. CF-7 cells and MDA-MB-231 cells were seeded out on E-Plates and allowed to settle. Cells were stimulated with MNF. Cellular proliferation and adherence was measured every 15 minutes using xCELLigence RTCA station. The serum-starved cells were incubated with various amounts of (R,R′)-MNF concentrations for 24 hours and the levels of [3H]thymidine incorporation were measured. The results are shown in FIG. 7. MNF inhibited the incorporation of radiolabelled thymidine in both CF-7 cells and MDA-MB-231 cells.

### Example 7

The effect of AM251 (a GPR55 agonist) and ICI 118,551 (a (2-AR antagonist) pretreatment on the inhibitory activity of (R,R′)-MNF was tested in both CF-7 cells and MDA-MB-231 cells. The cells were serum-starved, were incubated for 24 hours with either MNF alone, MNF+AM251, AN251 alone, MNF+ICI 118,551, or ICI 118,551 alone, after which the levels of [3H]thymidine incorporation were measured. The results are shown in FIG. 8. The data indicates that the inhibitory effect of MNF is blocked by GPR55 agonist, AM251 but not by β2-AR antagonist, ICI 118,551.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

What is claimed is:

1. A method of reducing the expression of Multidrug resistance protein (Mdr) in tumor cells comprising:
administering an effective Mdr expression-reducing amount of a pharmaceutical composition containing a fenoterol analogue and a pharmaceutically acceptable carrier to a patient having a tumor that includes cancer cells expressing ß2-adrenergic receptor (AR), cannabinoid (CB) receptor and epidermal growth factor receptor (EGFR).

2. The method of claim 1 wherein the pharmaceutical composition administered contains one or more compounds selected from the group consisting of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol, and their respective stereoisomers.

3. The method of claim 1 wherein the pharmaceutical composition administered contains a compound of the formula:

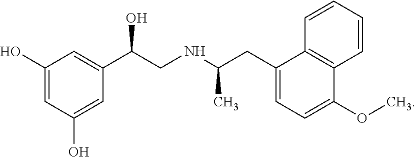 [SMILES: COc1ccc(C[C@@H](C)NC[C@H](O)c2cc(O)cc(O)c2)c2ccccc12]

4. The method of claim 2 further comprising administering a chemotherapeutic agent before, during or after administration of the pharmaceutical composition.
